(19) United States
(12) Patent Application Publication
(10) Pub. No.: US 2006/0292243 A1
(43) Pub. Date: Dec. 28, 2006

(54) Arsenic compounds for the treatment of the arsenic-sensitive blast-cell related diseases

(76) Inventor: Shao-Chi Hsin, Taipei (TW)

Correspondence Address: HERSHKOVITZ & ASSOCIATES, 2845 DUKE STREET, ALEXANDRIA, VA, 22314, US

(21) Appl. No.: 11/165,454

(22) Filed: Jun. 24, 2005

(51) Int. Cl. A61K 33/36

(52) U.S. Cl. 424/623

(57) ABSTRACT

A method of treatments for arsenic-sensitive blast-cell related diseases comprising administering a therapeutically effective amount of arsenic compounds to a human or an animal.

### BACKGROUND OF THE INVENTION

1. Field of Invention

The present invention is related to arsenic compounds for the arsenic-sensitive blast-cell related diseases, more particularly, the present invention is related to the treatment for malaria, Trypanosoma, bronchial asthma and systemic lupus erythematosus.

2. Description of the Related Art

Systemic Lupus Erythematosus

Systemic lupus erythematosus (SLE) is a systemic autoimmune disease with the potential to be directly involved in multiple organ systems. The clinical manifestations of SLE include skin rash and joint pain, and severe and progressive kidney involvement. SLE patients typically present elevated serum levels of antibodies to nuclear constituents (i.e., antinuclear antibodies). Specific T helper cells (Th cells) initiate and sustain the production of pathogenic, anti-nuclear autoantibodies during the onset and progression of systemic lupus erythematosus (SLE) through cognate interaction with autoimmune B cells. These SLE-associated Th cells are primarily responsible for driving the pathogenic autoimmune response. Without the help provided by these Th cells, autoimmune B cells are unable to produce the disease-causing (pathogenic)-autoantibodies associated with SLE. Some of the critical epitopes (i.e. autoantigenic determinants) to which these Th cells are directed have been localized to the histone proteins of nucleosomes (i.e. DNA-protein complexes in the nuclei of animal cells). For example, in lupus prone mice, SLE-associated autoepitopes have been identified at amino acid positions 10-33 of the H2B histone protein, at amino acid positions 85-102 of the H3 histone protein, and at amino acid positions 16-39 and 71-94 of the H4 histone protein. In order to study the disease, workers have employed several animal models, including the F 1 hybrid of New Zealand Black (NZB) and New Zealand White (NZW) mice, MRL mice homozygous for the lymphoproliferation (lpr) gene and BXSB mice, which carry the disease accelerating Yaa gene on the Y chromosome.

Several years ago, an animal model of SLE was established in the laboratory of one of the present inventors. This model, based on the concept of the idiotypic network, developed a wide spectrum of lupus-related autoantibodies and clinical manifestations. The induction was carried out by the immunization of mouse strains that do not develop any spontaneous autoimmune disorders, with a human anti-DNA monoclonal antibody (mAb) which bears a common idiotype termed 16/6 Id. Following immunization, the mice produced antibodies specific to the 16/6 Id, antibodies that bear the 16/6 Id and antibodies directed against different nuclear antigens (dsDNA, ssDNA, Sm, ribonucleoprotein (RNP), Ro, La and others). The serological findings were associated with leukopenia, elevated erythrocyte sedimentation rate, proteinuria, abundance of immune complexes in the kidneys and sclerosis of the glomeruli, which are typical manifestations of SLE. The present inventors have further shown that the experimental disease could be induced by a murine anti-16/6 Id mAb and by the mouse anti-anti 16/6 Id (16/6 Id+) mAb. The induction of the disease is genetically controlled, and thus is strain dependent. This unique model for the induction of experimental SLE provides the appropriate tools to clearly dissect the different steps and the linked immune parameters involved in the induction and development of SLE.

A majority of patients with SLE have symptoms of kidney failure. Clinical presentations typically include asymptomatic hematuria or proteinuria, acute nephritic or nephrotic syndromes, rapidly progressive glomerulonephritis and chronic renal insufficiency.

Current treatments have addressed lupus nephritis, although commonly used therapeutic regimes are potentially toxic and may be ineffective for some high risk patients. Typically, intensive immunosuppressive regimes are prescribed. For severe SLE, immunosuppressives such as chemotherapies and cyclosporin are used. Other treatments include treatment with corticosteroids and cytotoxic drugs. Alternative therapies include treatment with cyclophosphamide and prednisone. Side effects of long term use of prednisone include development of high blood pressure, diabetes and osteoporosis. Currently, many pharmaceutical companies are searching for alternative therapies. La Jolla Pharmaceutical Company is conducting phase II/III trials of LJP394 Toleragen, designed to target B cells that display anti-double stranded DNA antibodies that are implicated in kidney damage. Genelabs Technologies, Inc. is conducting a phase III trial of DHEA, a naturally occurring androgen, with the goal of overall disease reduction. Other drug therapies include IDEC-131, a humanized monoclonal antibody that targets CD40 on helper T cells and a 5G1.1 C5 complement inhibitor.

Malaria

Malaria is an infectious parasitic disease transmitted by mosquitoes, and is characterized by periodic fever and an enlarged spleen. Malaria affects some 200 million people a year. Malaria in humans is caused by 4 species of parasitic protozoa belonging to the genus Plasmodium. Of these, P. falciparum produces the severe disease while P. malariae, P. vivax and P. ovale cause milder forms.

Malaria is transmitted by infected female Anopheline mosquitoes. The Plasmodia parasite matures in the insect, and is then transferred when the mosquito bites a human. Inside the human, the parasite settles first in the liver, multiplies and then invades the red blood cells. This is when the symptoms of malaria become evident.

Despite numerous attempts at eradication, malaria remains a serious endemic disease in many areas of Africa, Latin America and Oceania, with a worldwide mortality rate of approximately 1 million per year. One of the major factors contributing to the continued presence of malaria is the emergence of malaria parasites that are resistant to one or more anti-malarial compounds.

Mefloquine is an anti-malarial compound which is effective against strains of the Plasmodium parasite which have developed resistance to conventional anti-malarial agents. However, mefloquine resistance has now been reported in a number of areas including areas of Thailand. Nevertheless, mefloquine is still one of the most effective anti-malarial mono-therapies and its use has increased greatly. Recently, the drug has attracted considerable adverse publicity owing to the incidence of severe neuropsychiatric side-effects, e.g. depression, psychosis, panic attacks, generalised anxiety. Although central nervous system (CNS) side-effects had been reported previously, their incidence had been regarded as sufficiently low to be of little concern. However, the widespread use of the drug by holidaymakers has resulted in a greatly increased number of CNS side-effect reports. A recent study about comparison of adverse events associated with use of mefloquine and combination of chloroquine and proguanil as antimalarial prophylaxis in which 3851 travellers taking prophylactic anti-malarial medication were surveyed, has confirmed that there is a significant excess of adverse neuropsychiatric events associated with mefloquine administration compared with an alternative prophylactic treatment (proguanil plus chloroquine).

Trypanosoma

The disease commonly known as sleeping sickness is caused by the parasite Trypanosoma brucei. This disease, which is transmitted to humans through the bite of the tsetse fly, has been estimated to affect 300-500 thousand people and maybe fatal if left untreated.

Trypanosoma brucei exists as two subspecies, Trypanosoma brucei rhodesiense and Trypanosoma brucei gambiense. Each form develops within the tsetse fly and the metacyclic trypomastigote of each form is passed into the bloodstream of its victim through the salivary gland of the fly following a bite. Once in the blood stream the parasite spreads rapidly throughout the host. The initial infection stage involves infection of the lymph system, and results in enlarged lymph nodes, headache and irregular fevers. The second stage involves invasion of the central nervous system with neurological symptoms of progressive mental apathy, extended daytime sleeping, and loss of appetite. These neurological effects along with concurrent involvement of the muscular system can result in progression to paralysis and irreversible coma.

The parasite's ability to invade the central nervous system has required two separate treatments due to the difficulty of drug penetration through the blood-brain barrier. Each of the current treatments requires drug delivery through injections. Intramuscular injections are required for either pentamidine isethionate or suramine sodium for the early stage of infection. The second stage of infection, involving the central nervous system, requires intravenous injections of either melarsoprol or eflornithine. These drugs result in serious side effects including hypotension, abdominal pain, vertigo, hypersalivation and mild nephrotoxicity for pentamindine isethionate treatment; and nausea, vomiting, uticaria and possible renal damage or exfoliative dermatitis when using suramine sodium. The development of new drugs for treating sleeping sickness has been very slow. For example, the commonly used second stage drug melarsoprol was developed in 1932 and is a highly toxic arsenic-based molecule. This drug can cause myocardial damage, hypertension, exfoliative dermatitis and reactive encephalopathy, which occurs in 5-10% of the patients and can lead to death. Eflornithine, an inhibitor of the enzyme ornithine decarboxylase, is the only drug suitable for patients where melarsoprol is ineffective but it is poorly effective against Trypanosome brucei rhodesiense. This drug causes mild side effects such as diarrhea, anemia, thrombocytopenia, vomiting and fever.

Recently, Trypanosoma brucei resistant strains to these drugs have been identified. For this reason and due to the few treatment options it is important to develop new therapeutic strategies for treating this disease. In an effort to identify a new and more effective method for treating Trypanosoma brucei Gelb et al. reported the successful use of prenyl transferase inhibitors in inhibiting the growth of Trypanosoma brucei parasites.

In view of the need to find new treatments for Trypanosoma brucei infections, those skilled in the art would welcome an effective method for the treatment utilizing inhibitors of Trypanosoma brucei prenyl transferases.

Bronchial Asthma

Bronchial asthma was regarded as an abnormality of respiratory smooth muscle in which afflicted individuals experienced the onset of bronchospasm as a consequence of over-reactivity of the bronchial smooth muscle for many years. However, the bronchial mast cell was thought to play a critical role in the stimulation of bronchial smooth muscle by producing leukotriene C4 (the slow-reacting substance of anaphylaxis) and histamine which cause contraction later. Over the past few years, a dramatic change in thinking regarding the pathophysiology of bronchial asthma has occurred and in this new appreciation of this disease, inflammation of the airway, particularly that caused by eosinophilic leukocytes, or “eosinophils,” has been suspected.

Eosinophils are a type of leukocyte containing cytoplasmic granules that stain strongly with acidic dyes. Eosinophils have been associated with bronchial asthma since the early part of this century and they are characteristically found in large numbers in the lung tissue of patients dying of asthma. In the mid 1970s, it was demonstrated that the severity of bronchial asthma can be related to the number of eosinophils in the peripheral blood of the patients. Also around this time, studies of eosinophils had shown the presence of basic (cationic) granule proteins. One of the principal proteins associated with eosinophil granules, the major basic protein (MBP), was so-named because in the guinea pig it comprises more than 50% of the granule protein, is strongly basic (arginine-rich), and is proteinaceous. Those findings were parallel to an early observation that this cell accumulates in the asthmatic lung.

The prominent role of eosinophils as effector cells in asthma and allergy has stimulated considerable interest in the mechanisms that are involved in the recruitment of these cells. Of particular importance are the chemical signals released in the lung that initiate and orchestrate the process of eosinophil recruitment from the blood microvessels in the airway wall. Some scientists lavaged guinea pig airways at various intervals after allergen challenge, injected the bronchoalveolar lavage (BAL) fluid intradermally in assay guinea pigs that were previously injected intravenously with ¹¹¹In-eosinophils, and measured eosinophil accumulation in the skin sites. Using this technique, eosinophil chemoattractant activity was detected in BAL fluid with a peak at 3 to 6 hr after challenge. This activity was purified in a series of high-performance liquid chromatography steps, using the in vivo skin bioassay at each stage in order to locate the chemoattractant. Microsequencing revealed a novel 73 amino acid C—C chemokine that is termed here as ‘eotaxin’ (condensed from eosinophil chemotaxin). The chemokine was present in three fractions at the final reversed phase high-performance liquid chromatography purification stage, which are believed to correspond to three glycosylation variants of eotaxin. The purified protein was potent in stimulating eosinophils in vitro and in vivo, but had no significant effect on neutrophils.

Guinea pig eotaxin was shown to be potent in inducing a calcium flux in human eosinophils, indicating the existence of a human homologue. Subsequently, primers based on the protein sequence have been used to clone cDNA for guinea pig, mouse, rat, horse and human eotaxins. All of the eotaxins are highly potent eosinophil chemoattractants, with greater than 60% protein sequence similarity, but with some functional cross-species restrictions (eg human eotaxin is inactive on guinea pig eosinophils, whereas human eotaxin is active in the rat, making this species useful for in vivo studies).

Nowadays, the small protein, eotaxin, is known to be synthesized by a number of different cell types, and is stimulated by interleukin-4 and interleukin-13, which are produced by T-helper (Th) 2 lymphocytes. If eotaxin amount can be reduced, stimulation by eotaxin will be prevented. This provides the potential for drugs that can prevent eosinophil recruitment into the lung and the associated damage and dysfunction.

### SUMMARY OF THE INVENTION

An aspect of the present invention is related to a method of treatments for the arsenic-sensitive blast-cell related diseases comprising administering a therapeutically effective amount of arsenic compounds to a human or an animal.

Preferably, said arsenic compound is administered parenterally.

Preferably, said arsenic-sensitive blast-cell related disease is selected from a group of diseases consisting of hypersensitive disease, immune or autoimmune disease, fibroblast-related disease, inflammation disease and parasitic disease.

More preferably, immune or autoimmune disease is connective tissue diseases, autoimmune thyroid disease, neuromuscular junction autoimmune disease, autoimmune gastrointestinal disease, autoimmune hepatitis, primary biliary cirrhosis, primary sclerosing cholangitis, autoimmune carditis or arteritis.

More preferably, said immune or autoimmune disease is systemic lupus erythematosus, rheumatoid arthritis, sclerosis, Graves disease, Myasthenia Gravis, multiple sclerosis, ulcerative colitis or Crohn disease.

Preferably, hypersensitive disease is bronchial asthma, hypersensitivity pneumonitis, diffuse pulmonary interstitial fibrosis, allergic rhinitis, eosinophil-associated nasal inflammation, vernal conjunctivitis and urticaria.

Preferably, inflammation disease is pneumoconiosis, osteomyelitis, leprous nodule, syphilis, tuberculosis, hepatitis, tumor formation, chronic obstructive pulmonary disease.

Preferably, said fibroblast-related disease is selected from a group of diseases consisting of hepatic fibrosis, pulmonary fibrosis, cutaneous and subcutaneous fibrosis and systemic fibrosis.

More preferably, said fibroblast-related disease is liver cirrhosis, pneumoconiosis, tuberculosis, severe acute respiratory syndrome (SARS), Adult (Acute) Respiratory Distress Syndrome (ARDS), chronic obstructive pulmonary disease (COPD), scarring, keloid, psoriasis, cystic fibrosis or neurofibromatosis.

Preferably, said parasitic disease is malaria or trypanosomiasis.

Preferably, said arsenic-sensitive blast-cell is blast-like cell of white blood cell, peripheral blood mononuclear cell, fibroblast, parasite, which is sensitive to said arsenic compound.

Preferably, the total amount administered is in the range of from about 0.001 μM to about 20 μM.

More preferably, the total amount administered is in the range of from about 0.1 μM to about 15 μM.

Most preferably, the total amount administered is in the range of from about 0.1 μM to about 10 μM.

Other objectives, advantages and novel features of the invention will become more apparent from the following detailed description when taken in conjunction with the accompanying drawings.

### BRIEF DESCRIPTION OF THE DRAWINGS

FIG. 1 is a bar chart showing the result of MTT assay for human fibroblasts exposed in arsenic trioxide; and

FIG. 2 is a bar chart showing the result of the biological effect of arsenic trioxide on cell cycle.

### DETAILED DESCRIPTION OF THE INVENTION

The present invention provides a method for treating inflammation diseases, comprising administering to a human or animal afflicted with such a disease, an amount of pharmaceutically acceptable arsenic compounds, wherein said amount is effective to counteract the symptoms of inflammation diseases.

The present invention provides a method for treating fibroblast-related diseases, comprising administering to a human or animal afflicted with such a disease, an amount of pharmaceutically acceptable arsenic compounds, wherein said amount is effective to counteract the symptoms of fibroblast-related diseases.

The present invention provides a method for treating malaria and trypanosomiasis, as well as other parasitic diseases, comprising administering to a human or animal afflicted with such a disease, an amount of pharmaceutically acceptable arsenic compounds, wherein said amount is effective to counteract the symptoms of malaria, trypanosomiasis or other diseases.

The present invention provides a method for treating systemic lupus erythematosus, as well as other immune or autoimmune diseases, comprising administering to a human or animal afflicted with such a disease, an amount of pharmaceutically acceptable arsenic compounds, wherein said amount is effective to counteract the symptoms of systemic lupus erythematosus or other diseases.

The present invention provides a method for treating bronchial asthma, as well as other hypersensitivity diseases, comprising administering to a human or animal afflicted with such a disease, an amount of pharmaceutically acceptable arsenic compounds, wherein said amount is effective to counteract the symptoms of bronchial asthma or other diseases. It is believed that the arsenic compounds can reduce the eotaxin amount and decrease the eosinophils accumulation on the affected parts of the human or animal. A preferred method of administering the arsenic compounds is parenterally, as by injection, infusion, topical application or inhalation. Thus, the present method provides a therapy for bronchial asthma in the lung, eosinophil-associated intranasal inflammation, including inflammation of the paranasal sinuses, and eosinophil-associated inflammation of the eye, such as vernal conjunctivitis. Preferred arsenic compounds are inorganic arsenic compounds, inclusive of arsenic trioxide (As₂O₃) and arsenic hexoxide (As₄O₆).

The present method is believed to involve counteracting or preventing the symptomologies caused by eosinophil via the parenteral administration of arsenic compounds to the afflicted or susceptible human or animal. For example, the present invention provides a therapy for bronchial asthma and the other hypersensitivity diseases of the respiratory tract, by intravenous injection or infusion of an arsenic compound, which is preferably arsenic trioxide. The arsenic compounds in turn are able to decrease eosinophil amount in situ. Topical administration of arsenic compounds, e.g., in eyedrops, can relieve the symptoms of conditions due to eosinophil-associated inflammation of the eye, such as vernal conjunctivitis.

The present invention also provides a unit dosage form comprising an amount of arsenic compounds optimal to treat bronchial asthma of human and animal by some conversion methods.

The present invention demonstrates that arsenic compounds are effective in treating asthmatic disease because they do reduce the hyperactivity to brochoconstriction. When asthma attacks, increasing in airway resistance is a major pathophysiologic phenomenon. Tests of the effect of the arsenic trioxide on the increase in respiratory resistance (bronchospasm) due to methacholine administration to BALB/c mice showed that arsenic trioxide does decrease the airway responsiveness.

The term “arsenic-sensitive blast-cell” used herein refers to a cell that is sensitive to arsenic compound. The cell may be leukocyte, lyphocyte, mast cell, T cell, B cell, hemopoietic stem cell or fibroblastic cells.

The term “arsenic-sensitive blast-cell related disease” used herein refers to hypersensitive disease, immune or autoimmune disease, fibroblast-related disease, inflammation disease or parasitic disease.

This invention is illustrated further rather than limited by the following examples. All of the references listed in the application are hereby incorporated by reference.

### EXAMPLES

### Example 1

### Arsenic Trioxide Induces Apoptosis of T cells and Peripheral Blood Mononuclear Cells in Vitro

Arsenic trioxide (Asadin®) induces differentiation and apoptosis of malignant cells in vitro and in vivo and has been used in the treatment of a variety of hematological malignancies, the main goal of this example is to try to identify the apoptotic effect of arsenic trioxide on various PBMCs (peripheral blood mononuclear cells), and the methodologies used in this invention include:

1. The isolation procedure using Ficoll Plaque for exploiting the different densities of RBCs and PBMCs to separate them.

2. The detection of apoptosis by PI staining through flowcytometry.

3. The detection of apoptosis within different subsets of PBMCs by the combination of mAb-fluorescein tandem staining and the treatment of Phiphilux through flowcytometry. Phiphilux is a substrate of caspase-3, composed of a specific oligo-peptide with its sequence subjected to functional caspase-3 cutting and, at each end of the peptide, is covalently coupled to two identical fluorophores.

In the PI-based study, under different concentrations of arsenic trioxide (0, 0.5, 2.5, 5, or 10 μM), the percentages of cell death (within all PBMCs) in 24 hr and 48 hr were calculated respectively in Table 1.1.

TABLE 1.1
the percentages of cell death in 24 hr and 48 hr

| Concentration | 24 hr % cell death | 48 hr % cell death |
|---|---|---|
| Control | 6.2 ± 0.6% | 7.3 ± 0.8% |
| 0.5 μM | 7.3 ± 1.2% | 7.6 ± 0.6% |
| 2.5 μM | 13.7 ± 1.6% | 15.2 ± 2.3% |
| 5.0 μM | 14.6 ± 2.1% | 17.2 ± 1.9% |
| 10 μM | 17.8 ± 1.8% | 20.3 ± 2.6% |

There was no dose-dependent relationship from 2.5 to 10 μM in 24 or 48 hr (however, there was indeed a slight increase in the death level for the 48 hr group), so some specific types of PBMCs might be more sensitive to arsenic trioxide than others.

Furthermore, according to that suspect, the apoptotic cell death within subsets of PBMCs was investigated by detecting caspase-3 activity within gated PBMC groups according to their surface markers (i.e. for T lymphocytes were recognize as “CD3⁺”, B lymphocytes as “CD19⁺”), the results show that most T cells (96%) are dead when treated with 5 μM arsenic trioxide for 48 hour (Table 1.2). Thus, the conclusion is that T lymphocytes are more sensitive to arsenic trioxide according the flow data.

TABLE 1.2
Levels of T cell apoptosis (%) after 48 hr treatment

| | |
|---|---|
| Control | 34.5 |
| 1 μM | 36.1 |
| 5 μM | 96.2 |

### Example 2

### Biological Effect of Arsenic Trioxide on Cultured Human Fibroblasts Showed Highlighted on Cell Cycle and Arsenic Compound Treated Fibroblasts Underwent Both Apoptosis and Necrosis

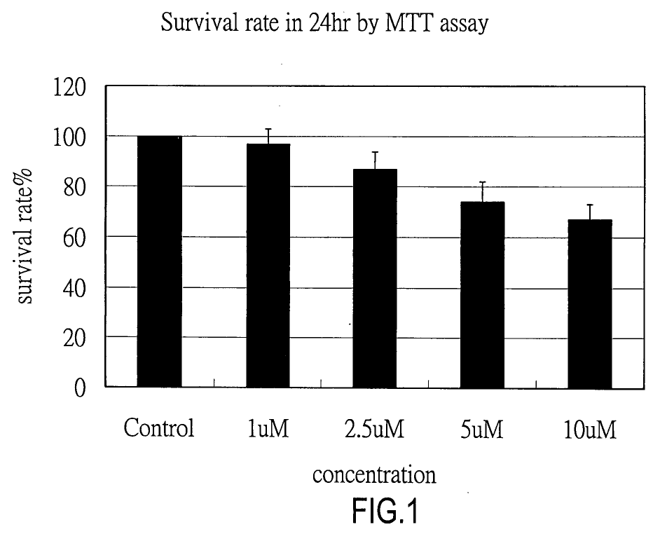

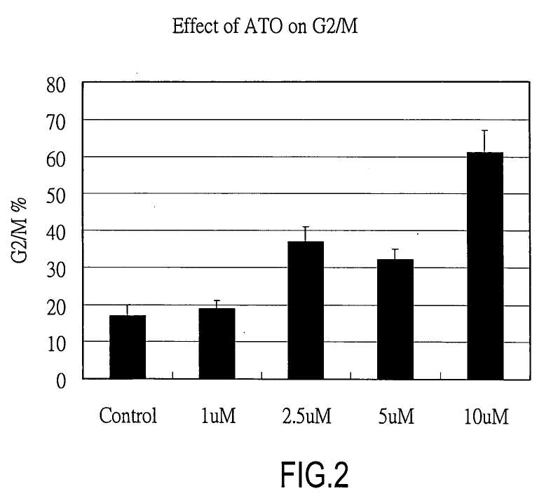

Arsenic trioxide has been approved for APL (acute promyelocytic leukemia) therapy, and though much work remains to be done on the application of arsenic trioxide and the mechanism of arsenic toxicity is not fully understood, several recent works pointed out the involvement of oxidative stress in arsenic-induced DNA damage in living cells may cause cell cycle arrest. In cultured human fibroblasts exposed for 24 hr to 1 to 10 μM arsenic trioxide and the fibroblasts were assayed by MTT assay, flowcytometry and western blot and promoter leuciferase assay to find out the biological effect of arsenic trioxide on cell cycle of cultured human fibroblasts, and though it was found that arsenic trioxide can cause fibroblasts cell cycle arrest especially in G2/M, on the molecular level, p53 seems to be bound with cell cycle arrest caused by arsenic trioxide (FIGS. 1 and 2).

Fibroblasts were treated with different concentrations of ATO for 24 hours, cells were fixed and the apoptotic bodies were detected by DAPI staining. The data showed that there were no obvious apoptosis in control, 1 μM and 2.5 μM. In contrast, there were obvious apoptotic bodies present in 5 μM and in the condition of 10 μM, fibroblasts underwent both apoptosis and necrosis.

### Example 3

### Method for the Treatment of Asthma and Like Hypersensitivity Diseases by Administration of Arsenic Compounds

3.1 Materials and Methods

BALB/c mice (female, 6-8 weeks) were immunized with ovalbumin (OVA) at day 0, 14, 21 and 28. 38 days later, animals were injected with arsenic trioxide (ATO) i.p. for 7 days and antigen challenges with OVA were performed at day 42, 43, 44. At day 45, the airway enhanced pause (PenH) values were measured. 48 hours after the last antigen challenges, the mice were sacrificed, the trachea cannulated, and bronchoalveolar lavage (BAL) was performed by four repeated lavages with balanced salt solution (HBSS) injected into the lungs via the trachea. Total cell counts were performed with a hemocytometer after staining with trypan blue, and then cytospins were prepared, and a differential count of 300 cells per slide was performed. To obtain the absolute number of each leukocyte subtype in the lavage, the percentage of each cell type was multiplied by the total number of cells that were recovered from the bronchoalveolar lavage fluid (BALF). Eotaxin in the BALF was measured via ELISA. Four groups of mice were treated as Table 3.1.

Group A mice were challenged by PBS only and then treated by PBS, too. Group B mice were immunized by ovalbumin and then treated by PBS only. Group C mice were immunized by ovalbumin and then treated by arsenic trioxide in a dosage of 2.5 mg per kg for mice. Group D mice were challenged by ovalbumin as group B and C and then treated by arsenic trioxide in a dosage of 5 mg/kg.

TABLE 3.1

| | Antigen challenge | Treatment |
|---|---|---|
| Group A | PBS | PBS |
| Group B | OVA | PBS |
| Group C | OVA | ATO 2.5 mg/kg |
| Group D | OVA | ATO 5 mg/kg |

3.2. Results
3.2.1 OVA Stimulation Index

After antigen challenges, mice serum were prepared and OVA-specific IgE values in serum were detected for evidence of immunization. The data in Table 3.2 show the mice of group B, C, and D were successfully immunized by ovalbumin because the serum levels of OVA-specific IgE were significantly elevated in comparison with mice in group A, the control group.

TABLE 3.2
OVA-specific IgE (SI.) in serum

| | Mean | SD |
|---|---|---|
| Group A | 0.30 | 0.09 |
| Group B | 2.06 | 0.81 |
| Group C | 2.20 | 0.70 |
| Group D | 1.96 | 0.31 |

3.2.3 Eotaxin in BALF

Table 3.3 shows the mean value and standard deviation of eotaxin in BALF of mice within each group. It was demonstrated that the arsenic treatment does diminish the eotaxin level after antigen challenge especially in a dosage of 2.5 mg/kg, because there is a marked and meaningful difference between group B and group C (p<0.05).

TABLE 3.3
mean value and standard deviation of eotaxin (pg/ml) in BALF of
mice within each group

| | Mean | SD |
|---|---|---|
| Group A | 14.42 | 8.39 |
| Group B | 20.90 | 4.97 |
| Group C | 12.38* | 3.92 |
| Group D | 16.00 | 4.06 |

*p < 0.05, compare with group B (OVA-PBS)

3.2.3. Eosinophils in BALF

The white blood cells in BALF were counted and differentiated. The mean concentration and standard deviation of each cell type in each group was shown in the Table 3.4. Every kind of white cell was increased in immunized groups when in comparison with the non-immunized group. However, arsenic trioxide treatments (group C and D) do diminish significantly the eosinophil number but elevate the macrophage and neutrophil numbers in contrast with non-treatment group (group B). It can be concluded that eosinophil will be potently reduced by arsenic trioxide treatment and the result is consistent with eotaxin level in BALF.

TABLE 3.4
mean concentration and standard deviation of each cell type in each
group (×1000 cells/ml)

| | Macrophage | Eosinophil | Neutrophil | Lymphocyte* |
|---|---|---|---|---|
| Group A | 135.6 ± 54.5^{#} | 3.2 ± 2.1 | 8.4 ± | 12.9 ± |
| | | | 4.9 | 8.2 |
| Group B | 243.8 ± 69.3 | 105.3 ± 60.7 | 77.1 ± | 37.8 ± |
| | | | 32.8 | 16.3 |
| Group C | 621.0 ± 199.6 | 28.6 ± 13.0 | 200.7 ± | 37.0 ± |
| | | | 75.2 | 9.4 |
| Group D | 499.1 ± 131.6 | 33.2 ± 12.5 | 244.0 ± | 28.7 ± |
| | | | 72.6 | 13.6 |

*Basophil count is so negligible that the numbers are not shown.
^{#}Values given as mean ± SD

3.2.4. Airway Responsiveness in Arsenic Treated Animal

The mice were challenged with methacholine (Mch.) in an increasing concentration and the airway constriction of the mice was measured by using pulmonary function test, whereafter the data of Table 3.5 was obtained. This data shows that airway hyper-reactivity of mice was successfully blocked by arsenic trioxide treatment especially in a dosage of 2.5 mg/kg, when comparing the data between group B and group C. The result is also consistent with the results of eotaxin and eosinophil in BALF.

TABLE 5
the mice in an increasing concentration and the result of the airway
constriction of the mice by pulmonary function test

| | Mch(mg/ml) | | | |
|---|---|---|---|---|
| | 6.25 | 12.5 | 25 | 50 |
| Group A | 2.20 ± 0.28^{#} | 8.22 ± 1.22 | 11.99 ± 1.50 | 14.21 ± |
| | | | | 1.06 |
| Group B | 2.50 ± 0.40 | 8.00 ± 0.93 | 14.52 ± 1.42 | 19.15 ± |
| | | | | 3.35 |
| Group C | 3.62 ± 0.47 | 7.83 ± 1.13 | 10.15 ± 0.84* | 9.83 ± |
| | | | | 1.17* |
| Group D | 2.55 ± 0.35 | 8.82 ± 0.88 | 11.57 ± 1.52 | 12.51 ± |
| | | | | 1.90 |

^{#}Values given as mean ± SD
*p < 0.05, compare with Group B (OVA-PBS)

### Example 4

### Method for the Treatment of Systemic Lupus Erythematosus and Like Autoimmune Diseases by Administration of Arsenic Compounds

4.1 Arsenic Trioxide Preparation

Commercial arsenic trioxide injection solution (Asadin®, 10 mg/10 ml/vial) was chosen and diluted by PBS solution.

4.2 Anti-human Systemic Lupus Erythematosus Activity in Vivo

In vivo studies on the activity of arsenic compound were carried out by using an experimental animal model of human Systemic Lupus Erythematosus. NZB/NZW denotes the mouse strain that spontaneously develops an autoimmune syndrome having notable similarities to human Systemic Lupus Erythematosus. Autoimmune disease in NZB/NZW mice is characterized by antinuclear antibodies (IgG) production about 4 months of age, and glomerulonephritis with proteinuria is developed when at 9 months of age, and that is the major cause of death (uremia).

At 24 weeks of age, mice (3 groups) were treated 3 times per week with PBS (Group I as positive control), 2.5 mg/kg of As₂O₃ (Group II), or 5 mg/kg of As₂O₃ (Group III) administered intraperitoneally. The treatment was stopped at 28 weeks of age. Mice were observed daily for clinical signs of disease and for mortality and were bled every 4 weeks for determination of anti-DNA antibody production.

Standard ELISA assay to measure the serum levels of anti-DNA antibodies was performed as follows. The plates were coated 10 μg/mL mBSA 4° C. overnight before coating dsDNA and ssDNA diluted with PBS to concentration of 7.5 μg/mL. ssDNA was prepared by boiling for 20 min then placed on ice immediately for 20 min. After overnight incubation at 4° C., the plates were washed and blocked with gelatin-PBS solution. The serum was diluted and then added to the appropriate wells, which were then incubated at 37° C. for 45 min. Horseradish peroxidase-comjugated anti-mouse γ-chain-specific antibodies were added. After incubation at 37° C. for 50 min, then room temperature for 10 min, 2,2′-azino-bisC3-ethylbenzthiazoline-6-sulphonic acid (ABTS) solution was used as substrate and the absorbance was measured at 405 nm. The level of anti-DNA IgG is presented as ELISA units (EU/ml) compared with mAb 10F10. The OD value generated by 74 ng/ml of 10F10 Ab was defined as 1 EU/ml for anti-DNA IgG. The level of anti-nucleosome IgG was calculated as EU by comparison with 10F10 Ab. The absorbance value generated by 231 ng/ml of 10F10 Ab was defined as 1 EU/ml of anti-nucleosome IgG. The results of anti-dsDNA are shown in Table 4.1 and the results of anti-dsDNA are shown in Table 4.2.

TABLE 4.1
IgG Anti-ssDNA titer (ELISA Unit)

| | | Time after treatment | | |
|---|---|---|---|---|
| | Before | (weeks) | | |
| Treatment | treatment | 4 | 8 | 12 |
| PBS | 0.171 | 0.333 | 0.465 | 0.105 |
| 2.5 mg/kg ATO | 0.155 | 0.347 | 0.400 | 0.105 |
| 5 mg/kg ATO | 0.176 | 0.303 | 0.296 | 0.201 |

TABLE 4.2
IgG Anti-dsDNA titer (ELISA Unit)

| | | Time after treatment | | |
|---|---|---|---|---|
| | Before | (weeks) | | |
| Treatment | treatment | 4 | 8 | 12 |
| PBS | 0.280 | 0.623 | 0.679 | 0.381 |
| 2.5 mg/kg ATO | 0.262 | 0.572 | 0.464 | 0.442 |
| 5 mg/kg ATO | 0.362 | 0.285 | 0.357 | 0.702 |

As shown in Table 4.1 and Table 4.2, sera obtained from mice treated with arsenic trioxide showed significantly lower anti-DNA antibody levels compared to mice treated with PBS alone. A higher concentration of arsenic trioxide solution had more effect than lower concentration. Because anti-DNA antibody levels are strongly correlated with SLE disease activity, these results demonstrate that administration of arsenic trioxide provides a new approach for the treatment of human SLE.

Urine protein levels were determined by colorimetric analysis using dipsticks (Multistix®, Bayer). Severe glomerulonephritis was defined as proteinuria more than 1 g/L (2+) by urine dipstick. The results are shown in Table 4.3.

TABLE 4.3
Mice with severe glomerulonephritis (%)

| Treatment | | Time (weeks) |
|---|---|---|
| | 37 | 40 |
| PBS | 25 | 100 |
| 2.5 mg/kg ATO | 16.67 | 40 |
| 5 mg/kg ATO | 25 | 25 |

Data reported in Table 4.3 show that a marked delay in the onset of severe proteinuria was achieved in animals treated with 2.5 mg/kg ATO and 5 mg/kg ATO in comparison with untreated animals. Whereas although 100% of control mice had developed proteinuria, some As₂O₃-treated mice did not develop proteinuria, and a significant percentage of these mice maintained normal renal function without evidence of proteinuria. Treatment with arsenic trioxide induced an evident reduction in the glomerulonephropathic progression, in comparison to the control group.

Coincident with ameliorating the clinical signs of severe immune complex nephritis, a dramatic prolongation of survival was observed. The results are shown in Table 4.4.

TABLE 4.4
survival rate (%) of the mice treated with As₂O₃

| | Time (weeks) | | | | | | | |
|---|---|---|---|---|---|---|---|---|
| Treatment | 0 | 22 | 31 | 38 | 39 | 40 | 41 | 42 |
| PBS | 100 | 100 | 66.7 | 50 | 50 | 16.7 | 0 | 0 |
| 2.5 mg/kg | 100 | 100 | 100 | 83.3 | 66.7 | 66.7 | 66.7 | 50 |
| ATO | | | | | | | | |
| 5 mg/kg | 100 | 100 | 66.7 | 66.7 | 66.7 | 66.7 | 66.7 | 66.7 |
| ATO | | | | | | | | |

Data reported in Table 4.4 show that 50% of arsenic trioxide treated mice were still alive after 43 weeks, while no animals treated with placebo (PBS) were still alive.

### Example 5

### Arsenic Compounds for the Treatment of Parasitic Diseases

5.1 Arsenic Trioxide Preparation

Commercial arsenic trioxide injection solution (Asadin®, 10 mg/10 ml/vial) was chosen. The molecular weight of As₂O₃ is 197.84.

5.2 Malaria Inhibition Test

FCC1/NH strain (from Hainan, P.R.C.) of P. falciparum was used. The strain of the parasite was cultured by culture media mainly composed of RPMI-1640(GBCO), HEPES (GBCO), NaHCO₃, and AB type serum. In a given experiment, Petri dish cultures were diluted with culture medium containing an amount of noninfected type B human erythrocytes to obtain a culture with a final parasitemia of about 1%. The resulting culture was ready for addition to microtitration plates with 96 flat-bottom wells.

The final volume added to each of the 96-well microtitration plates was 200 μl, consisted of 20 μl of complete medium with or without the drug (arsenic trioxide) and 180 μl of the parasitized culture. The microtitration plates were incubated in 5% CO₂ for an additional 48 hr, at 37 C. The suspensions were discarded and infection rate of RBC in each well was calculated by microscope examination after Giemsa staining. When drugs interfere, an inhibitory dose of 50% (IC₅₀) and 90% (IC₉₀) can be calculated. A series of experiments is shown below.

5.2.1

The malaria inhibition experiment was performed in 4 lanes for each arsenic trioxide dose, and the initial infectious rate is 1.8%. After 48 hours of incubation, 5000 RBCs in each well were checked by microscope. The inhibition rate is shown in Table 5.1.

TABLE 5.1
Inhibition rate to malaria

| | Log₁₀ of | | Inhibition | Probability |
|---|---|---|---|---|
| Dose | Dose | Infection rate | rate | unit |
| (uM) | (X) | (%) | (%) | (Y) |
| 0.25 | −0.6021 | 42.3 | 57.7 | 5.1942 |
| 0.5 | −0.3010 | 24.5 | 75.5 | 5.6903 |
| 1.0 | 0 | 19.5 | 80.5 | 5.8596 |
| 2.0 | 0.3010 | 13.7 | 86.3 | 6.0939 |
| 4.0 | 0.6021 | 9.3 | 90.7 | 6.3225 |
| 8.0 | 0.9031 | 6.8 | 93.2 | 6.4909 |

Y = 0.8177x + 5.8188
r² = 0.9651
IC₅₀ = 0.10 uM = 19.78 ng/ml 95% confidence limit (11.43˜34.2 ng/ml)
IC₉₀ = 3.68 uM = 728.05 ng/ml 95% confidence limit (420.84˜1259.53 ng/ml)

5.2.2

Another malaria inhibition experiment was performed in 4 lanes for each arsenic trioxide dose, and the initial infectious rate is 1.5%. After 48 hours of incubation, 5000 RBCs in each well were checked by microscope. The inhibition rate is shown in Table 5.2.

TABLE 5.2
Inhibition rate to malaria

| | Log₁₀ of | Infection | Inhibition | Probability |
|---|---|---|---|---|
| Dose | Dose | rate | rate | unit |
| (uM) | (X) | (%) | (%) | (Y) |
| 0.031 | −1.509 | 79.9 | 20.1 | 4.1619 |
| 0.063 | −1.201 | 73.7 | 26.3 | 4.3659 |
| 0.125 | −0.903 | 68.3 | 31.7 | 4.5239 |
| 0.25 | −0.6021 | 54.9 | 45.1 | 4.8743 |
| 0.5 | −0.3010 | 44.4 | 55.6 | 5.1408 |
| 1.0 | 0 | 17.9 | 82.1 | 5.9192 |
| 2.0 | 0.3010 | 12.9 | 87.1 | 6.1311 |
| 4.0 | 0.6021 | 11.2 | 88.8 | 6.2160 |
| 8.0 | 0.9031 | 5.1 | 94.9 | 6.6352 |

Y = 1.0904x + 5.6582
r² = 0.9706
IC₅₀ = 0.25 uM = 49.28 ng/ml 95% confidence limit ((28.00˜86.73 ng/ml)
IC₉₀ = 3.73 uM = 737.94 ng/ml 95% confidence limit (419.28˜1298.77 ng/ml)

5.2.3

The other malaria inhibition experiment was performed in 12 lanes for each arsenic trioxide dose, and the initial infectious rate is 1.1%. After 48 hours of incubation, 5000 RBCs in each well were checked by microscope. The inhibition rate is shown in Table 5.3.

TABLE 5.3
Inhibition rate to malaria

| | Log₁₀ of | Infection | Inhibition | Probability |
|---|---|---|---|---|
| Dose | Dose | rate | rate | unit |
| (uM) | (X) | (%) | (%) | (Y) |
| 0.5 | −0.3010 | 27.4 | 72.6 | 5.6008 |
| 1.0 | 0 | 12.9 | 87.1 | 5.1311 |
| 2.0 | 0.3010 | 8.5 | 91.5 | 6.3722 |
| 4.0 | 0.6021 | 3.6 | 96.3 | 6.7866 |
| 8.0 | 0.9031 | 2.0 | 98.0 | 7.0537 |

Y = 1.1832x + 6.0328
r² = 0.9849
IC₅₀ = 0.13 uM = 25.72 ng/ml 95% confidence limit (18.91˜34.9 ng/ml)
IC₉₀ = 0.96 uM = 189.93 ng/ml 95% confidence limit (139.65˜258.3 ng/ml)

5.2.4

The other malaria inhibition experiment was performed in 6 lanes for each arsenic trioxide dose, and the initial infectious rate is 1.1%. After 48 hours of incubation, 5000 RBCs in each well were checked by microscope. The inhibition rate is shown in Table 5.4.

TABLE 5.4
Inhibition rate to malaria

| | Log₁₀ | | Inhibition | Probability |
|---|---|---|---|---|
| Dose | of Dose | Infection rate | rate | unit |
| (uM) | (X) | (%) | (%) | (Y) |
| 0.5 | −0.3010 | 24.5 | 75.5 | 5.6903 |
| 1.0 | 0 | 15.7 | 84.3 | 6.0027 |
| 2.0 | 0.3010 | 9.0 | 91.0 | 6.3408 |
| 4.0 | 0.6021 | 4.0 | 96.0 | 6.7507 |
| 8.0 | 0.9031 | 1.9 | 98.1 | 7.0749 |

Y = 1.5032x + 5.8726
r² = 0.9495
IC₅₀ = 0.32 uM = 63.31 ng/ml 95% confidence limit (40.32˜99.4 ng/ml)
IC₉₀ = 2.09 uM = 413.48 ng/ml 95% confidence limit (263.06˜258.30 ng/ml)

5.3 Anti-Trypanosoma Test

Arsenic trioxide (Asadin®, 10 mg/10 ml/vial) was used to check the toxicity on the Trypanosoma brucei. Viable cell count was microscopically monitored. The complete killing effect over 7 days was observed at dose of 0.1 mg/ml/day for 3 days. One dose regimen of 0.3 mg/ml showed less killing potency at least one log difference than 3 doses regimen. This is one third of recommended dosage of Melarsoprol (an Anti-Trypanosoma drug). Moreover, cytology observation noticed specific death morphology on cell toxicity. Cells became thread-like instead of the usual small, round death cell. Further cytology examination on the death cells will be examined. In conclusion, arsenic trioxide can kill cultured Trypanosoma brucei at dosage comparable, if not better, to that of Melarsoprol.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method of treatments of arsenic-sensitive blast-cell related diseases comprising administering a therapeutically effective amount of arsenic compound to a human or an animal.

2. The method as claimed in claim 1, wherein said arsenic compound is administered parenterally.

3. The method as claimed in claim 1, wherein said arsenic-sensitive blast-cell related disease is selected from a group of diseases consisting of hypersensitive disease, immune or autoimmune disease, fibroblast-related disease, inflammation disease and parasitic disease.

4. The method as claimed in claim 3, wherein said immune or autoimmune disease is connective tissue diseases, autoimmune thyroid disease, neuromuscular junction autoimmune disease, autoimmune gastrointestinal disease, autoimmune hepatitis, primary biliary cirrhosis, primary sclerosing cholangitis, autoimmune carditis or arteritis.

5. The method as claimed in claim 4, wherein said immune or autoimmune disease is systemic lupus erythematosus, rheumatoid arthritis, sclerosis, Graves disease, Myasthenia Gravis, multiple sclerosis, ulcerative colitis or Crohn disease.

6. The method as claimed in claim 3, wherein said hypersensitive disease is bronchial asthma, hypersensitivity pneumonitis, diffuse pulmonary interstitial fibrosis, allergic rhinitis, eosinophil-associated nasal inflammation, vernal conjunctivitis and urticaria.

7. The method as claimed in claim 3, wherein said inflammation disease is pneumoconiosis, osteomyelitis, leprous nodule, syphilis, tuberculosis, hepatitis, tumor formation, chronic obstructive pulmonary disease.

8. The method as claimed in claim 3, wherein said fibroblast-related disease is selected from a group consisting of hepatic fibrosis, pulmonary fibrosis, cutaneous and subcutaneous fibrosis and systemic fibrosis.

9. The method as claimed in claim 8, wherein said fibroblast-related disease is liver cirrhosis, pneumoconiosis, tuberculosis, severe acute respiratory syndrome (SARS), Adult (Acute) Respiratory Distress Syndrome(ARDS), chronic obstructive pulmonary disease (COPD), scarring, keloid, psoriasis, cystic fibrosis or neurofibromatosis.

10. The method as claimed in claim 3, wherein said parasitic disease is malaria or trypanosomiasis.

11. The method as claimed in claim 1, wherein said arsenic-sensitive blast-cell is selected from a group consisting of leukocyte, peripheral blood mononuclear cell, fibroblast and parasite, which is sensitive to said arsenic compound.

12. The method as claimed in claim 1, wherein the total amount administered is in the range of from about 0.001 μM to about 20 μM.

13. The method as claimed in claim 12, wherein the total amount administered is in the range of about 0.1 μM to about 15 μM.

14. The method as claimed in claim 13, wherein the total amount administered is in the range of about 0.1 μM to about 10 μM.
